(19) United States
(12) Patent Application Publication
(10) Pub. No.: US 2009/0011981 A1
(43) Pub. Date: Jan. 8, 2009

(54) Novel Inhibitors of Angiogenesis

(75) Inventors: Jozef Van Damme, Brussel (BE); Paul Proost, Lubbeek (BE); Sofie Struyf, Wilsele (BE); Robert Strieter, Charlottesville, VA (US); Marie D. Burdick, Charlottesville, VA (US)

Correspondence Address: CLARK & ELBING LLP, 101 FEDERAL STREET, BOSTON, MA, 02110, US

(73) Assignees: K.U. LEUVEN RESEARCH & DEVELOPMENT, Leuven (BE); THE REGENTS OF THE UNIVERSITY OF CALIFORNIA, Oakland, CA (US)

(21) Appl. No.: 11/662,994

(22) PCT Filed: Aug. 15, 2005
(86) PCT No.: PCT/BE05/00140

§ 371 Date: Mar. 15, 2007

(30) Foreign Application Priority Data
Sep. 15, 2004 (GB) 0420517.5
Sep. 16, 2004 (GB) 0420606.6

(51) Int. Cl. A61K 38/16; A61K 38/08; C12P 21/00; C07K 7/08; A61P 35/00; C07K 14/00; C07K 7/06; A61K 38/10

(52) U.S. Cl. 514/12; 514/15; 514/14; 435/71.1; 530/328; 530/327; 530/326; 530/325; 530/324

(57) ABSTRACT

The present invention provides novel inhibitors of angiogenesis. The invention relates to PF4var1 and fragments and modifications thereof with anti-angiogenic activity. Therefore, the present invention relates to the use of said proteins and peptides as a medicine, more in particular for the treatment or prevention of angiogenesis or for the manufacture of a medicament for the prevention or treatment of angiogenic diseases. The invention also provides pharmaceutical compositions comprising said proteins and peptides and method of preventing or treating angiogenic disorders.

### FIELD OF THE INVENTION

The present invention provides inhibitors of angiogenesis. The invention relates to proteins and peptides with angiostatic activity. Therefore, the present invention relates to the use of said proteins and peptides as a medicine, more in particular for the treatment or prevention of angiogenesis or for the manufacture of a medicament for the prevention or treatment of angiogenic diseases and/or diseases involving undesired angiogenesis. The invention provides also pharmaceutical compositions comprising said proteins and peptides and methods of preventing or treating angiogenic disorders.

### BACKGROUND OF THE INVENTION

Angiogenesis involves the formation of new blood vessels of capillary origin and this phenomenon is tightly controlled by a set of factors that include Fibroblast Growth Factor (FGF-2) and Vascular Endothelial Growth Factor (VEGF). Angiogenesis constitutes an important process in the developing foetus and growing human, while in healthy adults, angiogenesis occurs significantly only during wound healing and in the menstrual cycle. It is now widely recognized that much of the angiogenic activity occurring in adults is pathological in nature. For example, proliferation of vascular endothelial cells and formation of new capillaries is essential for growth of solid tumors beyond a few cubic millimeters in volume.

Pathological angiogenesis occurs in many diseases of which cancer is one of the most important. Anti-angiogenic products are therefore highly needed. Many anti-angiogenic products have already been identified and are still being investigated. Protamine was one of the first inhibitors of endothelial cell growth. Also angiostatin, endostatin, thrombospondin-1, the 16-kD human prolactin fragment and Platelet Factor 4 have widely been studied for their anti-angiogenic activity.

Platelet Factor 4 (PF4/CXCL4) belongs to the CXC-chemokine family. The chemokine family consists of pro-inflammatory cytokines, primarily involved in chemoattraction and activation of specific leukocytes in various immuno-inflammatory responses [Rot et al. (2004) Annu Rev Immunol 22, 891-928; Kunkel (1999) J Clin Invest 104, 1333-1334; Mantovani (1999) Immunol Today 20, 254-257; Weber (2003) J Mol Med 81, 4-19]. It is known that chemokines activate blood platelets, important components of hemostasis, contributing to wound healing by forming thrombi and to the initiation of repair. However, a study has indicated that circulating activated platelets and platelet-leukocyte aggregates promote formation of atherosclerotic lesions. Finally, chemokines influence tumor growth by regulating angiogenesis. Net angiogenesis is determined by a balance between angiogenic and angiostatic factors within the local microenvironment. The CXC chemokine family is unique, as it comprises both angiogenic and angiostatic chemokines [Salcedo & Oppenheim (2003) Microcirculation 10, 359-370; Strieter et al. (1995) J Biol Chem 270, 27348-27357; Belperio et al. (2000) J Leukoc Biol 68, 1-8].

Platelet Factor 4 was the first chemokine described as a regulator of angiogenesis [Malone et al. (1990) Science 247, 77-79] and was previously also known as oncostatin, as described in U.S. Pat. No. 4,645,828. This angiostatic platelet-derived chemokine has been the subject of extensive research as a candidate anti-cancer drug [Bikfalvi & Gimenez-Gallego (2004) Semin Thromb Hemost 30, 137-144] and also clinical trials have already been performed with recombinant PF4 (rPF4) [Belman et al. (1996) Invest. New Drugs 14(4), 387-389.]. Many fragments, mutated forms and conjugates of PF4 have also already been investigated for their anti-angiogenic activity. Fragments have been described which are naturally occurring such as PF4¹⁷⁻⁷⁰, as described in EP0589719 or which are obtained by different approaches of protein engineering such as PF4 C-terminal fragment (PF4⁴⁷⁻⁷⁰). Malone et al. (cited above) described that also certain mutated forms of PF4 and C-terminal fragments thereof with no heparin-binding property exhibit anti-angiogenic activity. Certain publications describe that the 24 C-terminal amino acids contribute to the inhibitory activity of PF4 on angiogenesis, while WO93/13794 and WO 93/02192 describe that synthetic peptides corresponding to the carboxyterminal 13 amino acids of PF4 and mutants thereof inhibit angiogenesis. Interestingly, most of the publications on c-terminal PF4 fragments and mutations thereof retain the sequence Pro(58)Leu(59)Tyr(60) as well as Leu(67), suggesting that this motif is critical for angiogenic activity. PF4 fragments of at least 6 amino acids corresponding to AA20-26 of PF4 with angiogenic properties have also been described, such as in WO92/13874.

The molecular mechanism for PF4 angiostatic function is still a matter of debate. It has been suggested that PF4 is a unique chemokine that does not bind to a G protein coupled receptor (GPCR), but activates cells (i.e. neutrophils) and platelets through binding to cell surface glycosaminoglycans (GAG). The fact that GAG-binding is important for PF4 interaction is supported by another study, demonstrating that PF4 and CXCL10 share a GAG binding site on endothelial cells through which these chemokines inhibit FGF-induced endothelial cell growth. However, it is not clear whether PF4-binding to GAG alone is both necessary and sufficient to trigger endothelial cell signalling. PF4 function is not abrogated in heparan sulfate-deficient cells, and PF4 mutants or peptides lacking heparin-affinity are capable of inhibiting. A splice variant of CXCR3, designated CXCR3B, was identified that binds PF4. Finally, others have reported that the inhibitory effect of PF4 is mediated through complex formation with bFGF or CXCL8.

Although a lot of research has already been performed regarding PF4, important issues remain to be resolved, like an increased potency of PF4 fragments or analogues, an increase of stability or the best mode of action [Malone et al. cited above]. For example, WO9313794 describes that very high doses of PF4 are required when PF4 is used systemically for treatment of tumors.

More than a decade ago, PF4alt/PF4var1, the gene of a non-allelic PF4 variant was identified [Green et al. (1989) Mol Cell Biol 9, 1445-1451; Eisman et al. (1990) Blood 76, 336-344]. This variant of PF4 has never been extensively investigated for its biological activity. WO90/08824 describes the use of the PF4 variant protein as an immunostimulant in an immunosuppressed mouse model. The treatment of arthritis and other inflammatory diseases using PF4 or PF4 variant is described in WO94/07524.

### SUMMARY OF THE INVENTION

The present invention describes the isolation of natural PF4var1 protein from thrombin-stimulated platelets and demonstrates that the PF4var1 gene is translated into biologically active protein. It was determined that PF4var1 binds to heparin with lower affinity than PF4 on the basis that it eluted at lower salt concentrations from the heparin-Sepharose column. PF4var1 was characterised as a potent inhibitor of angiogenesis, which is more effective than PF4.

The present invention relates to the use of PF4var1, fragments, and modified versions of PF4var1 and PF4var1 fragments as a medicine, more in particular for the prevention and/or reduction of angiogenesis, more particularly for the treatment or prevention of angiogenic disorders or diseases involving angiogenic disorders or pathological angiogenesis and for the treatment and prevention of undesired angiogenesis. The present invention relates therefore to the use of PF4var1, fragments or modified versions of PF4var1 and PF4var1 fragments for the manufacture of a medicament for the prevention or treatment of diseases involving pathological angiogenesis. The invention also relates to pharmaceutical compositions comprising PF4var1, fragments or derivatives thereof and to methods of preventing or treating angiogenic disorders by using PF4var1, fragments or derivatives thereof. The invention also relates to processes for obtaining the PF4var1, fragments or derivatives thereof. Furthermore, the present invention relates to novel peptides having a certain sequence identity or sequence similarity to all or parts of PF4var1 and to novel fragments of PF4var1 and to their use as anti-angiogenic molecules.

Thus, the first aspect of the invention relates to the use of PF4var1 or and fragments of PF4var1 and modified versions thereof in the manufacture of a medicament for the prevention or inhibition of angiogenesis.

According to a particular embodiment of the invention relates to the use of a protein or peptide comprising the amino acid sequence Xaa-Xaa-Xaa-Xaa-Xaa-Xaa-Xaa-Glu-His corresponding to the X₆EH motif, wherein at least three of said 6 Xaa are identical to the corresponding amino acids in the sequence Leu-Leu-Tyr-Lys-Lys-Ile-Ile-Lys-Glu-His [SEQ ID NO: 5], in the manufacture of a medicament for the inhibition or reduction of angiogenesis. More particularly, the protein or polypeptide comprises the sequence Leu-Xaa-Xaa-Xaa-Xaa-Xaa-Xaa-Xaa-Glu-His [SEQ ID NO: 4] corresponding to the LX₇EH motif which is present in the carboxyterminal part of PF4var1 (amino acids 58, 66 and 67 in SEQ ID NO: 1 (FIG. 1). Even more particularly the protein or peptide has at least 60% sequence similarity to the sequence Leu-Leu-Tyr-Lys-Lys-Ile-Ile-Lys-Glu-His [SEQ ID NO: 5], such as, but not limited to a protein or peptide comprising the sequence Leu-Leu-Tyr-Lys-Lys-Ile-Ile-Lys-Glu-His depicted in SEQ ID NO: 5. In yet a further particular embodiment of the invention the protein or peptide comprises the sequence Leu-Leu-Tyr-Lys-Lys-Ile-Ile-Lys-Glu-His-Leu-Glu-Ser depicted in SEQ ID NO: 3. In yet a further embodiment of the present invention said protein or peptide comprises a sequence corresponding to a c-terminal fragment of PF4var1, or a fragment having at least 70% sequence identity therewith. In yet another embodiment of the invention the protein or peptide for use in the manufacture of the medicament according to the invention comprises the sequence of the PF4var1⁴⁷⁻⁷⁰ fragment, more particularly the sequence of the PF4var1⁶⁸⁻⁷⁰ fragment. A specific embodiment of the present invention relates to the use of a protein comprising the sequence of PF4var1 depicted in SEQ ID NO:1 in the manufacture of an anti-angiogenic medicament or in the prevention, reduction or inhibition of angiogenesis, most particularly pathological angiogenesis.

A more particular embodiment relates to peptides or proteins comprising the a sequence which is similar to modified compared to the C-terminal sequence of PF4var1, such as the sequence Leu-Leu*-Tyr-Lys-Lys-Ile-Ile-Lys-Glu-His [SEQ ID NO:5], wherein Leu*, Tyr, Lys and Ile are conservatively substituted. The conservative substitutions according to this embodiment are depicted in Table 1. Particular embodiments of such substitutions are represented by the sequence motif L-[VILM]-[YFW]-[KHR]-[KHR]-[VILM]-[VILM]-[KHR]-E-H. Another particular embodiment relates to a peptide with the X₆EH sequence motif comprising the X-K-K-X2-K-E-H motif or alternatively in which the one, two or three of the lysines residues in the peptide motif -X-K-K-X2-K-E-H are changed into either Gln or Glu. Yet another embodiment of the present invention envisages the use of a protein or peptide, wherein said protein or peptide comprises the sequence Leu-Leu-Tyr-Gln-Glu-Ile-Ile-Gln-Glu-His [SEQ ID NO: 6], more particularly comprising the sequence Leu-Leu-Tyr-Gln-Glu-Ile-Ile-Gln-Glu-His-Leu-Glu-Ser [SEQ ID NO: 20].

Specific embodiments of this aspect of the present invention include the use of the peptides selected from the group consisting of:
a peptide consisting of the sequence Leu-Leu-Tyr-Lys-Lys-Ile-Ile-Lys-Glu-His depicted in SEQ ID NO: 5;
a peptide consisting of the sequence Leu-Leu-Tyr-Lys-Lys-Ile-Ile-Lys-Glu-His-Leu-Glu-Ser depicted in SEQ ID NO: 3;
a peptide consisting of the sequence of the PF4var1⁴⁷⁻⁷⁰ fragment;
a peptide consisting of the sequence of the PF4var1⁶⁸⁻⁷⁰ fragment;
a peptide consisting of the sequence Leu-Leu-Tyr-Gln-Glu-Ile-Ile-Gln-Glu-His [SEQ ID NO: 6]
a peptide consisting of the sequence Leu-Leu-Tyr-Gln-Glu-Ile-Ile-Gln-Glu-His-Leu-Glu-Ser depicted in SEQ ID NO: 20; and
a peptide consisting of the sequence of PF4var1 depicted in SEQ ID NO:1.
   as anti-angiogenic agents or in the manufacture of an anti-angiogenic agent.

According to another aspect, the present invention provides for the use of the proteins and peptides as described above in the prevention or inhibition or reduction of angiogenesis, more particularly pathological angiogenesis. Most particularly, said pathological angiogenesis is associated with a disease, such as cancer, more particularly cancer of the prostate, lung, breast, bladder, kidney, pancreas, liver, ovaries, uterus, stomach or is a metastatic skin or rectal tumor. According to a particular embodiment the anti-angiogenic factors of the present invention are used for the reduction of tumor size and/or metastasis. According to an alternative embodiment the proteins and peptides as described above in the prevention or treatment of an eye disease involving pathological angiogenesis such as ischemic retinopathy.

Thus, the present invention provides a method of preventing or treating disease involving an angiogenic disorder comprising the step of administering to a mammal an effective amount of a protein or peptide comprising an amino acid sequence corresponding to the sequence Leu-Xaa-Xaa-Xaa-Xaa-Xaa-Xaa-Xaa-Glu-His [SEQ ID NO: 4] and having at least 60% sequence similarity to Leu-Leu-Tyr-Lys-Lys-Ile-Ile-Lys-Glu-His [SEQ ID NO: 5], or specific embodiments thereof as described above.

According to yet another aspect of the present invention relates to a process or method for obtaining PF4var1 or fragments thereof. The process comprises the stimulation of platelets to induce release of the platelet alpha-granules, e.g. with thrombin or ADP (adenosine diphosphate), or the production of platelet lysates, followed by a purification of PF4var1 out of the medium wherein the platelets were placed, as described herein. Fragments can be obtained by chemical or enzymatical cleavage, followed by purification and analysis. More particularly the present invention provides a process for obtaining PF4var1 or fragments thereof, which comprises the steps of—a) lysing platelets or stimulating platelets in a medium under conditions whereby the alpha-granules of the platelets are released in said medium and b) purifying PF4var1 from said medium, and optionally c) obtaining fragments from said PF4var1. Depending on the fragments involved, these can thus be obtained either directly from the medium or by cleavage of the (purified) PF4var1. In specific embodiments of this process, the platelets are stimulated with thrombin.

Yet another aspect of the invention provides for novel proteins and peptides with anti-angiogenic activity. More particularly, the present invention provides protein or peptides consisting of between 10 and 70 amino acids, more particularly between 11 and 13 amino acids, and alternatively about 20 or 30 amino acids and comprising a sequence of at least 10 amino acids which comprises the X₆EH motif. Most particularly the novel proteins and peptides of the present invention comprise the sequence Leu-Xaa-Xaa-Xaa-Xaa-Xaa-Xaa-Xaa-Glu-His [SEQ ID NO: 4] having at least 60% sequence similarity to the sequence Leu-Leu-Tyr-Lys-Lys-Ile-Ile-Lys-Glu-His [SEQ ID NO: 5]. Specific embodiments of the anti-angiogenic peptides of the present invention include proteins or peptides which comprises the sequence Leu-Leu-Tyr-Lys-Lys-Ile-Ile-Lys-Glu-His depicted in SEQ ID NO: 5, more particularly which comprises the sequence Leu-Leu-Tyr-Lys-Lys-Ile-Ile-Lys-Glu-His-Leu-Glu-Ser depicted in SEQ ID NO: 3.

A further specific embodiment of the present invention provides proteins or peptides which correspond to a c-terminal fragment of PF4var1, or to a fragment having at least 70% sequence identity therewith. More specific embodiments thereof relate to the proteins and peptides of the present invention comprising the sequence of the PF4var1⁴⁷⁻⁷⁰ fragment or the sequence of the PF4var1⁶⁸⁻⁷⁰ fragment. Alternative embodiments of the present invention include modified fragments or fragments of modified PF4var1 protein, such as proteins or peptides of between 10 and 69 amino acids comprising the sequence Leu-Leu*-Tyr-Lys-Lys-Ile-Ile-Lys-Glu-His [SEQ ID NO:5], wherein Leu*, Tyr, Lys and Ile are conservatively substituted. The conservative substitutions according to this embodiment are depicted in Table 1, herein. Particular embodiments of such substitutions are represented by the sequence motif L-[VILM]-[YFW]-[KHR]-[KHR]-[VILM]-[VILM]-[KHR]-E-H. Another particular embodiment is represented by a peptide in which the one, two or three of the lysines residues in the peptide motif L-X2-K-K-X2-K-E-H are changed into either Gln or Glu, such as the peptide comprising the sequence Leu-Leu-Tyr-Gln-Glu-Ile-Ile-Gln-Glu-His [SEQ ID NO: 6], more specifically comprising the sequence Leu-Leu-Tyr-Gln-Glu-Ile-Ile-Gln-Glu-His-Leu-Glu-Ser [SEQ ID NO: 20]. Specific embodiments of this aspect of the present invention include the proteins or peptides selected from:
a peptide consisting of the sequence Leu-Leu-Tyr-Lys-Lys-Ile-Ile-Lys-Glu-His depicted in SEQ ID NO: 5;
a peptide consisting of the sequence Leu-Leu-Tyr-Lys-Lys-Ile-Ile-Lys-Glu-His-Leu-Glu-Ser depicted in SEQ ID NO: 3;
a peptide consisting of the sequence of the PF4var1⁴⁷⁻⁷⁰ fragment;
a peptide consisting of the sequence of the PF4var1⁶⁸⁻⁷⁰ fragment;
a peptide consisting of the sequence Leu-Leu-Tyr-Gln-Glu-Ile-Ile-Gln-Glu-His [SEQ ID NO: 6]; and
a peptide consisting of the sequence Leu-Leu-Tyr-Gln-Glu-Ile-Ile-Gln-Glu-His-Leu-Glu-Ser depicted in SEQ ID NO: 20.

The present invention further relates to the use of the above described protein or peptides which correspond to fragments of PF4var1 or modifications thereof, as a medicament, more particularly as an anti-angiogenic agent.

Yet another aspect of the present invention relates to a pharmaceutical composition comprising PF4ᵥₐᵣ₁, fragments of PF4var1 or modified versions of PF4var1 or PF4var1 fragments comprising the X₆EH sequence and more particularly the LX₆EH, as an active ingredient in admixture with at least a pharmaceutically acceptable carrier, the said pharmaceutical composition being preferably intended for the prevention or treatment of angiogenic disorders such as cancer, in a mammal, specifically a human.

### DETAILED DESCRIPTION OF THE INVENTION

### Definitions

The term “angiogenesis” as used herein generally refers to the fundamental process by which new blood vessels are formed. The primary angiogenic period in humans takes place during the first three months of embryonic development but angiogenesis also occurs as a normal physiological process during periods of tissue growth, such as an increase in muscle or fat and during the menstrual cycle, wound repair and pregnancy.

The term ‘pathological angiogenesis’ as used herein refers to angiogenesis which is not beneficial to the health of the patient and is associated to a disease condition.

Similarly, an angiogenic disorder refers to a condition involving angiogenic dysfunctions, namely wherein an unwanted proliferation of capillary blood vessels or endothelial cells leading to pathological angiogenesis occurs. Pathological angiogenesis occurs in a number of diseases, such as in cancer (i.e. solid tumors, metastasis), aberrant wound repair, diabetic retinopathy, retrolental fibroplasia, neovascular glaucoma, psoriasis, angiofibromas, -immune and non-immune inflammation (including rheumatoid arthritis), capillary proliferation within atherosclerotic plaques, hemangiomas, endometriosis, Kaposi Sarcoma, etc.

The term ‘undesired angiogenesis’ as used herein refers to angiogenesis which in itself is not pathological but is, due to circumstances, undesired, e.g. physiological wound repair can be undesired in some circumstances.

The term ‘PF4var1’ refers to both the natural and recombinant versions of the human natural variant of the PF4 protein. The protein is also known as PF4alt, PF4V1, SCYB4V1, or CXCL4L1 (OMIM number 173461, accession numbers P10720 and M26167 at Swiss-Prot and Genbank databases, for the protein and genomic DNA sequence, respectively). The mature PF4var1 protein is obtained by processing of a longer propeptide. The 70 amino acid long version depicted in FIG. 1 and SEQ ID NO: 1 is the most abundant form of PF4var1 in platelet preparations. The numbering of the amino acids in this sequence is used for referral throughout this application. Also longer sequences, partially processed versions of the PF4var1 pro-protein, mostly having at their aminoterminus the 5 sequence Phe-Ala-Arg-Ala [SEQ ID NO: 17] occur in lower abundance in platelet preparations.

The ‘X₆EH’ motif as used herein refers to the sequence comprising the amino acids Glutamic acid and Histidine and, N-terminally thereof, a sequence of at least 7 amino acids of which at least 3 are identical to the sequence of M59 to AA65 of SEQ ID NO:1.

The ‘LX7EH motif’ as used herein refers to the sequence Leu-Xaa-Xaa-Xaa-Xaa-Xaa-Xaa-Xaa-Glu-His [SEQ ID NO: 4] wherein Xaa represents an unspecified amino acid. In the PF4var1 protein, the LX₇EH motif is present in the sequence Leu-Leu-Tyr-Lys-Lys-Ile-Ile-Lys-Glu-His [SEQ ID NO: 5].

A ‘fragment of PF4var1 as used herein refers to a protein or peptide having a sequence which is comprises least 10 amino acids and is shorter than the 70 amino acid sequence of SEQ ID NO:1, comprising at least the X₇EH motif and optionally the LX₇EH motif.

Amino acids are referred to herein with their full name, their three letter abbreviation or their one letter abbreviation (Table 1). Xaa stands for any amino acid, including both natural L-form or their D-form and unnatural amino acids such as amino acid analogues or other linker molecules that can be introduced in an amino acid chain to substitute amino acids.

TABLE 1
Classes of amino acids and their abbreviations

| | | 3 letter | 1 letter |
|---|---|---|---|
| Classes of amino acid | Full name | code | code |
| Nonpolar/hydrophobic | glycine | Gly | G |
| | alanine | Ala | A |
| | valine | Val | V |
| | leucine | Leu | L |
| | isoleucine | Ile | I |
| | methionine | Met | M |
| | phenylalanine | Phe | F |
| | tryptophan | Trp | W |
| | proline | Pro | P |
| Polar/hydrophilic | serine | Ser | S |
| | threonine | Thr | T |
| | cysteine | Cys | C |
| | tyrosine | Tyr | Y |
| | asparagine | Asn | N |
| | glutamine | Gln | Q |
| Negative charged | aspartic acid | Asp | D |
| | glutamic acid | Glu | E |
| Positive charged | lysine | Lys | K |
| | arginine | Arg | R |
| | histidine | His | H |

The term ‘modified version’ when referring to the PF4var1 protein, or a PF4var1 fragment refers to proteins or peptides wherein the X₆EH motif and/or the LX₇EH motif remains unchanged but wherein other amino acids are changed, deleted or inserted, compared to the natural PF4var1 protein. An amino acid change can be a so-called conservative substitution wherein the nature of an amino acid side chain is preserved. Conservative amino acids substitutions include substitutions within the classes depicted in Table 1 herein above.

The relation between a protein and a modified version can be expressed by the percentage of amino acid sequence similarity or amino acid sequence identity between two sequences. The percentage of amino acid or nucleotide sequence identity/similarity is determined by alignment of the two sequences and identification of the number of positions with identical/similar amino acids or nucleotides divided by the number of nucleotides or amino acids in the shorter of the sequences ×100. The alignment of two nucleotide sequences is performed by the algorithm as described by Wilbur and Lipmann (1983) Proc. Natl. Acad. Sci. U.S.A. 80:726, using a window size of 20 nucleotides, a word length of 4 nucleotides, and a gap penalty of 4. Two amino acids in a sequence alignment are similar of when they belong to a same class of amino acids in Table 1 above.

### DETAILED DESCRIPTION OF THE INVENTION

The current invention describes the anti-angiogenic properties of the non-allelic PF4 variant PF4var1 and modified versions thereof. The mature protein of PF4Var1 differs in only three amino acids located in the COOH-terminus (Pro58Leu, Lys66Glu and Leu67His) from PF4 [Green et al. cited above, Eisman et al. cited above]. The carboxyterminal region of PF4 contains a repeating structure of 2 basic lysines (including Lys65 and Lys66) followed by 2 hydrophobic residues. This variant has however never been shown to be a biologically active angiogenesis inhibitor. The present invention describes the isolation of natural PF4var1 protein from thrombin-stimulated platelets and demonstrates that the SCYB4V1 gene is translated into biologically active protein. This chemokine was found to be a potent inhibitor of angiogenesis, which is more effective than PF4.

Prevention and/or inhibition of angiogenesis is tested herein both in vitro and in vivo. In vitro inhibition of angiogenesis can be demonstrated based on inhibition of bFGF- or CXCL8-induced HMVEC chemotaxis. In vivo anti-angiogenic activity can be tested using the cornea micropocket assay, whereby angiogenesis is induced by bFGF. Inhibition or prevention of angiogenesis according to the present invention relates to inhibition of at least 10%, more particularly 20%, even more particularly more than 30-50%, most particularly more than 60% of the angiogenesis observed in the absence of the molecules of the present invention.

Thus the present invention relates to the use of PF4var1 and PF4var1 fragments as well as modified versions thereof comprising the X₆EH motif and/or the LX₇EH motif, for the inhibition of angiogenesis.

More particularly, the present invention relates to the use of PF4var1 and fragments and modified versions thereof in the inhibition or prevention of angiogenesis, more particularly in the treatment and/or prevention of diseases involving pathological angiogenesis or angiogenesis disorders.

According to a particular embodiment of the present invention, the pathological angiogenesis or angiogenic disorders that can be treated with the compound of the present invention are characterized by the formation and growth of blood vessels during the maintenance and the progression of a disease state, such as cancer. Many cells, tissues, structures or organs can be implicated in angiogenic disorders due to occurrence of angiogenesis within or comprising these cells, structures, organs or tissues. Examples of tissues where pathological angiogenesis is known to occur (and of the diseases associated therewith) include, but are not limited to the blood vessels (atherosclerosis, hemangioma, hemangioendothelioma), bone and joints (rheumatoid arthritis, synovitis, bone and cartilage destruction, osteomyelitis, pannus growth, osteophyte formation, neoplasms and metastasis), skin (warts, pyogenic granulomas, hair growth, Kaposi's sarcoma, scar keloids, allergic oedema, neoplasms), liver, kidney, lung, ear and other epithelia [inflammatory and infectious processes (including hepatitis, glomerulonephritis, pneumonia), asthma, nasal polyps, otitis, transplantation, liver regeneration, neoplasms and metastasis], uterus, ovary and placenta [dysfunctional uterine bleeding (due to intra-uterine contraceptive devices), follicular cyst formation, ovarian hyperstimulation syndrome, endometriosis, neoplasms], brain, nerves and eye (retinopathy of prematurity, diabetic retinopathy, choroidal and other intraocular disorders, leukomalacia, neoplasms and metastasis), heart and skeletal muscle (***pathological cardiac or skeletal muscle hypertrophy?), adipose tissue (obesity), endocrine organs (thyroiditis, thyroid enlargement, pancreas transplantation), hematopoiesis (AIDS (Kaposi), hematologic malignancies (leukemias, etc.), lymph vessels (tumor metastasis, lymphoproliferative disorders).

A specific embodiment of the invention relates to the use of PF4var1 and fragments and modified versions thereof in the treatment and/or prevention of eye diseases, such as, but not limited to ‘retinal ischemic diseases’, which generally refers to the condition whereby the retina's supply of blood and oxygen is decreased and whereby the peripheral portions of the retina lose their source of nutrition and stop functioning properly. Common diseases which lead to retinopathy are diabetic retinopathy, central retinal vein occlusion, stenosis of the carotid artery, and sickle cell retinopathy. Diabetic retinopathy is a major cause of visual loss in diabetic patients. In the ischemic retina the growth of new blood vessels occurs (neovascularisation). These vessels often grow on the surface of the retina, at the optic nerve, or in the front of the eye on the iris. The new vessels cannot replace the flow of necessary nutrients and, instead, can cause many problems such as vitreous haemorrhage, retinal detachment, and uncontrolled glaucoma. These problems occur because new vessels are fragile and are prone to bleed. If caught in its early stages, proliferative diabetic retinopathy can sometimes be arrested with panretinal photocoagulation. However, in some cases, vitrectomy surgery is the only option.

Another embodiment of the present invention relates to the use of PF4var1 and fragments and/or derivatives thereof in the treatment of cancer or tumor formation. By “tumor” it is meant a mass of abnormal tissue that arises without obvious cause from pre-existing body cells, has no purposeful function, and is characterized by a tendency to autonomous and unrestrained growth. Examples of tumors envisaged in the context of the present invention include but are not limited to prostate, lung, breast, rectal, bladder, kidney, pancreatic, liver, ovarian, uterine, metastatic skin, stomach or other tumors.

Current research suggests that each tumor arises from a single cell that has been transformed by one or more events. Such events include the activation of oncogenes and the absence or inactivation of specific tumor-suppressor genes. These transformed cells can form small clones, initially co-opting normal host vessels, growing to only several millimeters in size before their supply of nutrients becomes limited. At this point, the tumor may Ile dormant for prolonged periods (from months to years) until ultimately undergoing destruction by the immune system or switching to an angiogenic phenotype. This “switch” involves a shift in the local equilibrium between negative and positive endogenous regulators of angiogenesis. The tumor cells may achieve this shift in several ways, including the overexpression of angiogenic factors, the recruitment of host cells (such as macrophages) that can produce their own angiogenic factors, the mobilization of angiogenic proteins from the extracellular matrix (ECM), or a combination of these processes. If the production of proangiogenic factors is sufficiently robust, neighboring endothelial cells will be activated, leading to the sprouting of new capillaries.

Tumors are quite different from inflammatory or other swellings because the cells in tumors are abnormal in their appearance and other characteristics. Abnormal cells—the kind that generally make up tumors—differ from normal cells in having undergone one or more of the following alterations: (1) hypertrophy, or an increase in the size of individual cells; this feature is occasionally encountered in tumors but occurs commonly in other conditions; (2) hyperplasia or an increase in the number of cells within a given zone; in some instances it may constitute the only criterion of tumor formation; (3) anaplasia, or a regression of the physical characteristics of a cell toward a more primitive or undifferentiated type; this is an almost constant feature of malignant tumors, though it occurs in other instances both in health and in disease. In some instances the cells of a tumor are normal in appearance and are faithful reproductions of their parent types so that the differences between them and normal body cells are difficult to discern. Such tumors are also often benign. Other tumors are composed of cells that appear different from normal adult types in size, shape, and structure. They usually belong to tumors that are malignant. Such cells may be bizarre in form or be arranged in a distorted manner. In more extreme cases, the cells of malignant tumors are described as primitive, or undifferentiated, because they have lost the appearance and functions of the particular type of (normal) specialized cell that was their predecessor. As a rule, the less differentiated malignant tumor cells are, the more quickly that tumor may grow. Malignancy refers to the ability of a tumor to ultimately cause death. Any tumor, either benign or malignant in type, may produce death by local effects.

The common and more specific definition of malignancy implies an inherent tendency of the tumor's cells to metastasize (invade the body widely and become disseminated by subtle means) and eventually to kill the patient unless all the malignant cells can be eradicated. Metastasis is thus the outstanding characteristic of malignancy. Metastasis is the tendency of tumor cells to be carried from their site of origin by way of the circulatory system and other channels, which may eventually establish these cells in almost every tissue and organ of the body. The amount of new blood vessel growth can correlate with poor prognosis in several tumor types. Since the shedding of large numbers of tumor cells from the primary tumor may not begin until after the tumor has a sufficient network of blood vessels, angiogenesis may also correlate with metastatic potential. Destruction of the ECM is probably necessary to initiate the metastatic process. Microvessel density has been correlated with cancer invasion and metastasis in a number of human tumors including breast, prostate, lung, esophageal, colorectal, endometrial and cervical. In addition, bone marrow angiogenesis was found to be of prognostic value in patients with multiple myeloma and other hematopoietic malignancies.

In contrast to malignant tumor cells, the cells of a benign tumor invariably remain in contact with each other in one solid mass centred on the site of origin. Because of the physical continuity of benign tumor cells, they may be removed completely by surgery if the location is suitable. But the dissemination of malignant cells, each one individually possessing (through cell division) the ability to give rise to new masses of cells (new tumors) in new and distant sites, precludes complete eradication by a single surgical procedure in all but the earliest period of growth. A benign tumor may undergo malignant transformation, but the cause of such change is unknown. It is also possible for a malignant tumor to remain quiescent, mimicking a benign one clinically, for a long time. All benign tumors tend to remain localized at the site of origin. Many benign tumors are encapsulated. The capsule consists of connective tissue derived from the structures immediately surrounding the tumor.

Well-encapsulated tumors are not anchored to their surrounding tissues. These benign tumors enlarge by accretion, pushing aside the adjacent tissues without involving them intimately.

Among the major types of benign tumors are the following: lipomas, which are composed of fat cells; angiomas, which are composed of blood or lymphatic vessels; osteomas, which arise from bone; chondromas, which arise from cartilage; and adenomas, which arise from glands. For malignant tumors, examples comprise carcinomas (occur in epithelial tissues, which cover the body (the skin) and line the inner cavitary structures of organs (such as the breast, the respiratory and gastrointestinal tracts, the endocrine glands, and the genitourinary system). Sarcomas develop in connective tissues, including fibrous tissues, adipose (fat) tissues, muscle, blood vessels, bone, and cartilage. A cancer can also develop in both epithelial and connective tissue and is called a carcinosarcoma. Cancers of the blood-forming tissues (such as leukemias and lymphomas), tumors of nerve tissues (including the brain), and melanoma (a cancer of the pigmented skin cells) are classified separately.

According to an alternative embodiment, the present invention relates to the use of PF4var1 and fragments and modified versions thereof in the inhibition or prevention of undesired angiogenesis or physiological angiogenesis which is not desired. Examples thereof include the inhibition of wound-healing in some circumstances.

In a specific embodiment, the PF4var1 and/or fragments and/or modified versions thereof are used for the inhibition or prevention of angiogenesis, more particularly for the prevention and/or treatment of diseases involving angiogenic disorders in combination with any other therapy for the disease such as in the case for cancer, the therapies known in the art such as irradiation, chemotherapy or surgery.

According to a particular embodiment, the present invention relates to the use of PF4var1 with the sequence as depicted in SEQ ID NO: 1 as a medicine, more in particular for the treatment or prevention of a disease involving an angiogenic disorder. Results presented herein show that PF4var1 is much more potent than PF4 in the inhibition of angiogenesis in vivo.

According to a further particular embodiment the invention relates to the use of modified versions of PF4var1 in the treatment and/or prevention of diseases involving an angiogenic disorder. Based on the findings of the present invention it appears that the amino acids 66 (Glu) and 67 (His), more particularly amino acids 58 (Leu), 66 (Glu) and 67 (His) in PF4var1 have an important influence on the angiogenic activity of the protein. Thus, according to the present invention the modified versions of PF4var1 or fragments thereof comprise at least 10 amino acids comprising the X₆EH motif with 66 (Glu) and 67 (His), such as in the motif K-K-X2-K-E-H or the X2-I-I-X-EH motif, and most particularly retain the LX₇EH motif. Typically modified versions of PF4var1 or PF4var1 fragments additionally have an amino acid sequence similarity or identify of at least 60%, at least 70%, at least 80%, at least 90% with PF4var1 within the sequence corresponding to AA58 and M70 of PF4var1. Alternatively modified versions of PF4var1 or PF4var1 fragments additionally have an amino acid sequence similarity or a sequence identity of at least 60%, at least 70%, at least 80%, at least 90% with PF4var1 within the X₆EH or within the LX₇EH motif. Alternatively the similarity or identity between PF4var1 and the modified proteins and peptides of the present invention can be expressed as the number or amino acids (AA) that are similar or identical to the corresponding amino acids in the sequence of PF4var1. Thus, the present invention provides for proteins and peptides comprising the X₆EH motif or LX₇EH motif, whereby at least 3 M, more particularly at least 4, even more particularly at least 5, most particularly at least 6 AA (and for the LX₇EH motif most particularly at least 7 AA) are similar or identical to the corresponding sequence in PF4var1. According to a particular embodiment, the sequence similarity/identity over the entire length of the sequence of the modified versions of PF4var1 or fragments thereof is at least 70%, particularly 80% or higher, more particularly at least 90%, even more particularly at least 95%, most particularly at least 98% with PF4var1. According to a particular embodiment, the sequence of the modified PF4var1 comprising a sequence of at least 10 amino acids comprising the motif K-K-X2-K-E-H or the X2-I-I-X-EH motif, and additionally or alternatively comprising a Leucine at a position corresponding to M58 when compared to SEQ ID NO:1 (so as to comprise the LX₇EH motif, differs by the replacement of one or more of the X or Xaa amino acids by conservative substitutions according to Table 1. Another specific embodiment refers to a modified PF4var1 and/or modified PF4var1 fragments wherein on or more Lysine amino acids, are replaced by Glu or Gln. According to a further embodiment, the sequence of the modified PF4var1 comprising 66 (Glu) and 67 (His) in the X₆EH motif and optionally the LX₇EH motif differs by the replacement of one or more of the Xaa amino acids by non-conservative changes, for example with amino acids sharing structural analogy but belonging to a different class, as long as the modified versions retain their anti-angiogenic activity, which can be assessed through the methods described herein. According to the present invention, these modified versions of PF4var1 or fragments thereof can be used for the treatment of diseases involving angiogenic disorders or for the manufacture of a medicament for the prevention or treatment of diseases involving angiogenic disorders, such as cancer, angiogenic disorders of the eye, endometriosis, etc.

According to a particular embodiment, the present invention provides fragments of PF4var1 or modified versions thereof comprising the X₆EH motif, having anti-angiogenic activity. Particularly the fragments of the present invention comprise at least 10 amino acids, of which the X₆EH motif represents amino acid 3 to 10, most particularly fragments comprising the LX₇EH motif. Alternatively, the fragments comprise at least 10 amino acids of which the X₆EH motif represents amino acid 2 to 9 or 3 to 8. According to a more particular embodiment, said fragments have, within said sequence of 6 amino acids represented by X₆, at least 3, more particularly at least 4, most particularly at least 5 amino acids similar or identical to the corresponding amino acid sequence of SEQ ID NO: 1. Most particularly the PF4var1 fragments of the present invention are more active that PF4 or PF4 fragments. Additionally or alternatively the fragments of the invention retain at least 80% of the anti-angiogenic activity of PF4var1. Anti-angiogenic activity can be measured in different ways, more particularly using the assays described herein.

According to a particular embodiment the fragments comprise the carboxyterminus of PF4var1, i.e. the sequence Leu-Xaa-Xaa-Xaa-Xaa-Xaa-Xaa-Xaa-Glu-His-Leu-Glu-Ser [SEQ ID NO: 2]. A further particular embodiment of the invention relates to fragments of PF4var1 comprising the sequence Leu-Leu-Tyr-Lys-Lys-Ile-Ile-Lys-Glu-His-Leu-Glu-Ser [SEQ ID NO: 3]. The fragments can be of any length and are at least 10 amino acids long, but can be around 20, 25, 30, 35, or more. Therefore, a particular embodiment of the invention relates to the fragment of PF4var1 corresponding to Leu-Leu-Tyr-Lys-Lys-Ile-Ile-Lys-Glu-His-Leu-Glu-Ser [SEQ ID NO:3].

As detailed above, the invention also provides novel peptides comprising the motif Leu-Xaa-Xaa-Xaa-Xaa-Xaa-Xaa-Xaa-Glu-His [SEQ ID NO:4] and which are modified versions of fragments of PF4var1 and still have anti-angiogenic activity. A more particular embodiment relates to peptides or proteins comprising the sequence Leu-Leu*-Tyr-Lys-Lys-Ile-Ile-Lys-Glu-His [SEQ ID NO:5], wherein Leu*, Tyr, Lys and Ile are conservatively substituted or even non-conservative substitution is possible. A particular embodiment relates to the peptide in which the lysines in the peptide are changed into either Gln or Glu. Yet another particular embodiment relates to peptides comprising the sequence Leu-Leu-Tyr-Gln-Glu-Ile-Ile-Gln-Glu-His [SEQ ID NO: 6].

According to the present invention, these fragments and peptides can be used as a medicine, more in particular for the prevention or treatment of diseases involving angiogenic disorders or for the manufacture of a medicament for the prevention or treatment of diseases involving angiogenic disorders.

The present invention further relates to peptides and proteins comprising the sequence of PF4var1 or fragments thereof or modified version thereof as a fusion protein with another protein. Examples of such chimeric proteins envisaged include chimeric proteins consisting of at least a fragment of the C-terminal domain of PF4var1 as described herein and other proteins with a desirable therapeutic effect.

Yet another aspect of the invention relates to the use of the peptides and proteins comprising the sequence of PF4var1 or modified versions thereof such as Leu-Leu-Tyr-Lys-Lys-Ile-Ile-Lys-Glu-His-Leu-Glu-Ser [SEQ ID NO:3] as a diagnostic tool. Thus, for example the peptides of the present invention can be conjugated with for example fluorescent compounds such as fluoresceine, for diagnostic purposes, as described in EP0723015 for PF4 or fragments thereof.

Methods that can be used for the preparation of the proteins, peptides, fragments or mutants thereof are well known in the art.

PF4var1 can be obtained through a process of lysing platelets or stimulating platelets with thrombin, ADP (adenosin diphosphate), collagen, PAR (protease-activated receptor) 1-activating peptides, PAR4-activating peptides, TXA2 (thromboxane A2), thrombopoietin, plasmin, PAF (platelet activating factor) or combinations of these platelet activators. Subsequently, stimulated platelets release their alpha-granules in a medium, followed by a purification of PF4ᵥₐᵣ₁ out of the medium wherein the platelets were placed.

For fragments for example, starting from the isolated and purified PF4var1, fragments can be obtained by chemical or enzymatic cleavage of the protein and subsequent purification and analysis. Fragments, peptides or proteins can otherwise be prepared by using standard peptide synthesis chemistry including solid phase chemistry.

Peptides such as peptide fragments of PF4Var1 or modified versions thereof can be synthesized using conventional synthesis procedures commonly used by one skilled in the art. They can chemically be synthesised by manual synthesis or by using an automated peptide synthesizer (such as for example Model 433A, Applied Biosystems). Particularly, peptide synthesis is performed on a solid phase (resins) which are functionalised (i.e. with amines) to be able to couple amino acids in a stepwise way (solid phase peptide synthesis (SPPS)—contrary to solution phase peptide synthesis). A distinction can be made between t-Boc and Fmoc solid phase chemistry:

t-Boc SPPS: This was first introduced by Merrifield in 1963 (Merrifield, 1963). The concept of the SPPS consists of the following: the first protected amino acid is attached to an insoluble polystyrene solid support via an acid labile linker. The amino acids are protected by a temporary acid labile protecting group, i.e. t-butoxycarbonyl (t-Boc), on the a-amino position, and by a more acid stable benzyl type protecting group on the functionality of the side chain. The t-Boc group is deprotected by trifluoroacetic acid (TFA) followed by the neutralization and washing steps, and then the next protected amino acid couples to the amino peptide resin in the presence of activator. The deprotection and coupling steps are repeated until the desired sequence of the peptide is assembled. The final peptide is cleaved and deprotected from the resin simultaneously by liquid hydrogen fluoride which requires a special apparatus for its safe handling.

Fmoc-SPPS: The SPPS strategy with a temporary base labile a-amino protecting group, 9-fluorenylmethoxycarbonyl (Fmoc), was introduced by Carpino in 1972 (Carpino and Han, 1972). Generally speaking in Fmoc SPPS, a-amino group is protected by Fmoc and the side chain functionality is protected by the acid labile t-butyl type protecting groups. Fmoc-based SPPS provides an alternative to the t-Boc SPPS and offers the advantage of a milder acid cleavage process.

the different steps of Fmoc-SPPS are described hereunder:
Solid support: The SPPS requires a well-solvated gel to allow the reactions to take place between reagents in the mobile phase and functional groups on chains throughout the interior of a resin. The original resin was developed as a polystyrene polymer cross-linked with 1% of 1,3-divinylbenzene with a swelling capacity 3 fold in volume in DMF. A polyamide resin was introduced by Atherton and Sheppard (Atherton and Sheppard, 1989) under the concept that the solid support and peptide backbone should be of comparable polarities. Recently, resins based on grafting of polyethylene glycol (PEG) to low cross-linked polystyrene was developed such as Tentagel (Bayer and Rapp, 1986) and PEG-PS resins (Barany et al, 1992) with a swelling capacity 5 fold in volume in DMF. More recently, resins based on cross-linked PEG have also been available such as PEGA (Meldal M, 1992) and CLERA resins (Kempe and Barany, 1996) with a swelling capacity 11 and 6.5 fold in volume, respectively. Due to their excellent swelling property, Tentagel and PEGA resins have shown superior performance in peptide synthesis.
Linkers: The function of the linker is to provide a reversible linkage between the peptide chain and the solid support, and to protect the C-terminal a-carboxyl group. The commonly used resins to provide peptides acid are Wang, Hydroxymethyl-phenoxy acetyl (HMPA), Rink acid, 2-Chlorotrityl chloride, SASRIN. The most commonly used resin for peptide amide is Rink amide resin.
The first residue attachment: The esterification of the first amino acid to the hydroxyl group on the resin is one of the key steps to produce a high quality peptide. The incomplete loading and racemization will cause truncated and epimeric peptides respectively, as a result of slow esterification reaction. The commonly used loading methods are the HOBt active ester, symmetrical anhydride and dichlorobenzoyl chloride procedures.
Protecting groups: For routine synthesis, the global protecting strategy is employed to all reactive functionalities of the side chains. For instance, hydroxyl and carboxyl functionalities are protected by t-butyl group, lysine and tryptophan are protected by t-Boc group, and asparagines, glutamine, cysteine and histidine are protected by trityl group, and arginine is protected by the pbf group. A wide range of protecting groups are also available for different applications.
Fmoc deprotection: The removal of the Fmoc group is usually accomplished by treatment with 20-50% piperidine in DMF for 20 minutes. In the case of incomplete Fmoc deprotection, a stronger base such as 1,8-diazabicyclo[5.4.0]undec-7-ene (DBU) with 2% piperidine can be used.
Coupling: Amide bond formation involves activation of the carboxyl group of the amino acid. There are four major coupling techniques: (a) in situ coupling reagents such as carbodiimide-mediated coupling, BOP, HBTU as well as HATU, (b) preformed active esters such as Opfp, Osu, Onp, (c) preformed symmetrical anhydrides, (d) acid Halides such as acyl fluoride as well as acyl chloride.
Monitoring: The completion of the deprotection and coupling needs to be monitored to ensure the success of the SPPS. The most widely used monitoring reaction is the Ninhydrin test to examine the presence of free amino group as a result of incomplete coupling. Other methods such as the TNBS and the Chloranil test can be used as complementary methods to the Ninhydin test.
Cleavage and removal of the protecting groups: Fmoc SPPS is designed for simultaneous cleavage of the anchoring linkage and global deprotection of side-chain-protecting groups with TFA. The most commonly used cleavage cocktail is Reagent K (TFA/thioanisol/water/phenol/EDT: 82.5:5:5:5:2.5 v/v).
Peptide evaluation: Nowadays, the peptide quality is examined routinely by the analytical HPLC to determine the purity in conjunction with mass spectral analysis to determine the identity. Most of the crude peptides can be purified alone by the reversed phase HPLC to achieve the desired purity. The combinations of anion or cation HPLC purification followed by the reversed phase HPLC purification provide a powerful technique to purify a crude peptide with inferior quality. The peptide purity needs to be determined by analytical HPLC with two different buffer systems or even further by capilliary Electrophoresis (CE). Data from sequence analysis and amino acid analysis can provide further detailed informations on peptide homogeneity.
Peptide modifications: By using of orthogonal protecting group strategy, resins with novel linkers and customized cleavage protocols, modified peptides can be synthesized routinely. These modified peptides can be catagorized as biotinylated, branched, chromogenic, C-terminal modified, fatty acid containing, fluorescent, glycosylated, isoprenated, cyclic lactam, multiple disulfide, peptide mimetics, phosphorated and sulfation peptides.
Peptide ligation: The introduction of the ligation strategy (chemoselective coupling of two unprotected peptide fragments) by Kent (Schnolzer and Kent, 1992) provides the tremendous potential to achieve protein synthesis which is beyond the scope of SPPS. Many proteins with the size of 100-300 residues have been synthesized successfully by this method. Synthetic peptides have continued to play an ever increasing crucial role in the research fields of biochemistry, pharmacology, neurobiology, enzymology and molecular biology because of the enormous advances in the SPPS.

According to one embodiment of the invention peptides are prepared by Fmoc-solid phase peptide synthesis on a 433A automated peptide synthesizer (Applied Biosystems, Foster City, Calif.) using amino acids with 9-fluorenylmethyloxycarbonyl (Fmoc)-protected alfa-amino groups and a program applying conditional double coupling steps and intermediate capping of incomplete peptide chains with acetic anhydride. The following side-chain protecting groups for amino acids are used: tert-butyl for Ser, Thr and Tyr, tert-butyl ester for Asp and Glu, trityl for Asn, Cys and Gln, t-butyloxycarbonyl for Lys and 2,2,5,7,8,-pentamethylchroman-6-sulfonyl for Arg (all from Advanced ChemTech, Louisville, Ky.). The COOH-terminal Fmoc-protected amino acids can be linked to amino-functionalized resins such as p-hydroxymethyl-phenoxymethyl-polystyrene (HMP) resin (PE Biosystems) or Tentagel by for example a symmetrical anhydride binding or through activation with a mixture of HOBt/DIC (or PyBOP)/DIEA. The Fmoc group is cleaved from the peptide-resin (Fmoc-cleavage with 20% piperidine in DMF) and subsequent HBTU/HOBt/(DIEA)-activated Fmoc-protected amino acids are attached (4 eq. amino acid, 4 eq. activator reagent sometimes with addition of DIEA (diisopropylethylamine)). After each coupling step, incomplete peptide chains are capped with acetic anhydride [(CH₂CO)₂O/NMI (N-Me-imidazole)/pyridine]. Final deprotection and cleavage of the peptide from the resin is performed by incubating the synthesis product for 100 min at room temperature in the following cleavage mixture: 95% trifluoro acetic acid (TFA), 2.5% water and 2.5% thioanisol (and/or 1,2-ethanedithiol). The synthetic peptide is than separated from the resin on a medium porosity glass filter, precipitated into cold (4° C.) methyl t-butyl ether, centrifuged, washed, dissolved in ultrapure water and subsequently lyophilized. The peptides can be redissolved in water containing 0.1% TFA. The desired peptide can than be purified from incomplete fragments by RP-HPLC on a Resource RPC column (Amersham Pharmacia Biotech) by for example using as eluens 0.1% TFA and eluted in an water/acetonitrile gradient.

Alternatively, the peptides can be synthesized by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridization methodologies. Such DNA molecules may be incorporated into expression vectors, including plasmids, which are adapted for the expression of the DNA and production of the polypeptide in a suitable host such as bacterium, e.g., Escherichia coli, yeast cell, animal cell or plant cell.

The present invention provides potent anti-angiogenic factors which are naturally occurring molecules, i.e. which can be administered with limited averse reaction of the patient. The potency of the molecules of the invention allow the administration of lower dosages which may further result in a reduction of side effects.

The present invention demonstrates that PF4var1 has a more potent activity on angiogenesis than PF4, even a 100-fold higher potency. Accordingly, the dosages that can be used in order to prevent or treat angiogenic disorders using the proteins and peptides of the present invention can be reduced in an order of magnitude of 5,10, 20, 30, 50, 100 or more, in regard of doses used of PF4. According to experiments performed (FIGS. 4 and 5) chemotaxis of HMVEC toward bFGF or CXCL8 was inhibited by PF4var1 much more potently than by PF4. PF4var1 shows an inhibition of 50% of the migration of HMVEC at a concentration of around 2.5 ng/ml medium, while PF4 shows practically no inhibition of migration of HMVEC at concentrations as high as 300 ng/ml. FIG. 5 shows furthermore that at a concentration of 50 ng/ml PF4ᵥₐᵣ₁ inhibited almost for 100% the migration of the cells, while PF4 showed approximately an 80% inhibition. Also the in vivo data strongly support a much higher activity of PF4var1 than of PF4.

Dosage for systemic treatment: WO9313794 explains that for experiments wherein murine melanoma cells are injected intravenously in mice, optimal results in regard of inhibition of metastases or lung weight are obtained at high doses in excess of 5 mg PF4 per kg of body weight.

According to a particular embodiment of the present invention, the dose used to treat cancer by systemical administration of PF4var1 can for example be reduced compared to PF4, e.g. can be reduced to 0.5 mg, 0.25 mg or lower per kg body weight of PF4var1. For modified versions of PF4var1 or fragments thereof, doses can be calculated by accounting for the molecular weight of the modified versions or fragments, compared to the minimal dose needed of PF4 which is 5 mg/kg body weight as described in WO93/13794. Patients can be treated systemically with PF4var1 at dosages between 1 μg and 100 mg per kg per day, or between 1 μg and 50 mg per kg, or between 1 μg and 10 mg per kg, or between 1 μg and 1 mg per kg, or between 10 μg and 50 mg per kg or between 20 μg and 50 mg per kg or between 20 μg and 5 mg per kg body weight of the mammal treated therewith or also for analoguous doses of fragments of PF4var1 calculated by accounting for the molecular weight of the fragments.

For use in the prevention or treatment of angiogenic disorders and by different administration modes (i.e. intravenously, subcutaneously, intramuscularly as single or multiple dosages), dosages may be used in the range of 0.1 μg/kg to 200 mg/kg body weight, more in particular between 0.1 μg/kg and 20 mg/kg or 0.1 μg/kg and 10 mg/kg, or between 0.1 μg/kg and 4 mg/kg, or between, 0.5 μg/kg and 20 mg/kg or between 1 μg/kg and 10 mg/kg or between 1 μg/kg and 5 mg/kg or between 5 μg/kg and 5 mg/kg body weight of the mammal treated therewith.

Starting from the results of clinical trials performed with recombinant PF4 or from articles describing the use of PF4 in vivo such as from Kolber, Belman et al. (1996) Invest. New Drugs 14(4), 387-389, dosages needed of PF4var1 can be reduced and calculated.

Additionally or alternatively, the present invention provides for molecules which correspond to compounds naturally occurring in the body, more particularly the PF4var1 protein and fragments thereof, the administration of which represents a lower risk for adverse reactions of the patient. Accordingly the dosages that can be used in order to prevent or treat angiogenic disorders using the proteins and peptides of the present invention can be higher in regard of doses used of non-natural compounds (e.g. modified versions of PF4).

Compounds of the invention and their physiologically acceptable salts (hereafter collectively referred to as the active ingredients) may be administered by any route appropriate to the condition to be treated and appropriate for the compounds, here the proteins and fragments to be administered. Possible routes include regional, systemical, oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient. Regional treatment is useful for treatment of cancers in specific organs in the patient, including, but not limited to primary liver cancer, brain and kidney cancer and liver metastases from colon/rectal cancer. Treatment can be accomplished by intraarterial infusion. A catheter can be surgically or angiographically implanted to direct treatment to the affected organ, a subcutaneous portal, connected to the catheter can be used for chronic treatment, or an implantable, refillable pump may also be employed.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

The compositions used in these therapies may also be in a variety of forms. These include, for example, solid, semi-solid, and liquid dosage forms, such as tablets, pills, powders, liquid solutions or suspension, liposomes, suppositories, injectable and infusible solutions. The preferred form depends on the intended mode of administration and therapeutic application. The compositions also preferably include conventional pharmaceutically acceptable carriers and adjuvants which are known to those of skill in the art and which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986). Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness.

Preferably, the compositions of the invention are in the form of a unit dose and will usually be administered to the patient one or more times a day. PF4var1 or other compounds of the invention, may be administered to the patient in any pharmaceutically acceptable dosage form, including intravenous, intramuscular, intralesional, or subcutaneous injection. Moreover, gene therapy, such as viral-mediated gene therapy (such as described by Li et al. 2003, Cancer Biother & Radiother 18(5):829-840) with sequences encoding the PF4var1 protein or the fragments or the modified versions thereof, is also envisaged within the context of the present invention. It should, of course, be understood that the compositions and methods of this invention may be used in combination with other therapies, once improvement of the patient's condition has occurred, a maintenance dose is administered if necessary. Subsequently, the dosage or the frequency of administration, or both, may be reduced, as a function of the symptoms, to a level at which the improved condition is retained, When the symptoms have been alleviated to the desired level, treatment should cease. Patients may, however, require intermittent treatment on a long-term basis upon any recurrence of disease symptoms. It should be understood that the examples and embodiments described herein are for illustrative purposes only and that various modifications or changes in light thereof will be suggested to persons skilled in the art and are to be included within the spirit of this application and the scope of the appended claims.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound.

### BRIEF DESCRIPTION OF THE FIGURES

The following Examples, not intended to limit the invention to specific embodiments described, may be understood in conjunction with the accompanying Figures, incorporated herein by reference, in which:

FIG. 1: protein sequence of human PF4var1 [SEQ ID NO:1]

FIG. 2: purification of natural PF4var1 protein from stimulated platelets. Panel A: PF4 immunoreactivity; Panel B: SDS PAGE under reducing conditions of fractions 51-60 of Panel A. (left lane shows Mᵣ markers)

FIG. 3: Mass spectrometrical analysis of purified PF4var1 protein (fraction 54 of FIG. 1). The m/z values for the differently charged ions in the unprocessed spectrum (calculated from the average of 1500 spectra) are indicated, as are the numbers of protons they carry. Four different NH₂-terminal isoforms of PF4var1 are present, of which the NH₂-terminal sequence can be deduced based on the difference between the four proteins in the charge-deconvoluted spectrum (insert). Deduced NH₂-terminal sequences are indicated in the Table 2 herein.

FIG. 4: Inhibition of PF4var1 on bFGF-induced and CXCL8-induced endothelial cell chemotaxis. Panel A and B: capacity to induce migration of HMVEC by PF4var1 (1-100 ng/ml; open triangles) and of PF4 (1-300 ng/ml; black triangles). Panel A: Inhibition of chemotaxis of HMVEC towards bFGF (50 ng/ml) with different concentrations of PF4var1 (open squares) or PF4 (filled squares). Panel B: inhibition of chemotaxis of HMVEC towards CXCL8 (80 ng/ml) with different concentrations of PF4var1 (open circles) or PF4 (filled circles). Panel C: PF4var1 (50 ng/ml) and PF4 (50 ng/ml) reduce migration of HMVEC toward bFGF (50 ng/ml) or CXCL8 (80 ng/ml). Results (mean±SEM) are derived from 2 independent experiments in triplicate.

FIG. 5: Inhibition of in vivo angiogenesis of PF4var1 on bFGF-induced angiogenesis in a rat cornea micropocket assay. Panels are at 20× magnification.

FIG. 6: Effect of the local administration of PF4 and PF4var1 on tumor size reduction in vivo. A: evolution of tumor size in time upon administration of saline (lozenges), PF4var1 1 μg (squares) or PF4 5 μg (triangles); B: average tumor volume in mm³ after two weeks of treatment with saline (open block), PF4var1 1 μg (filled block) or PF4 5 μg (shaded block).

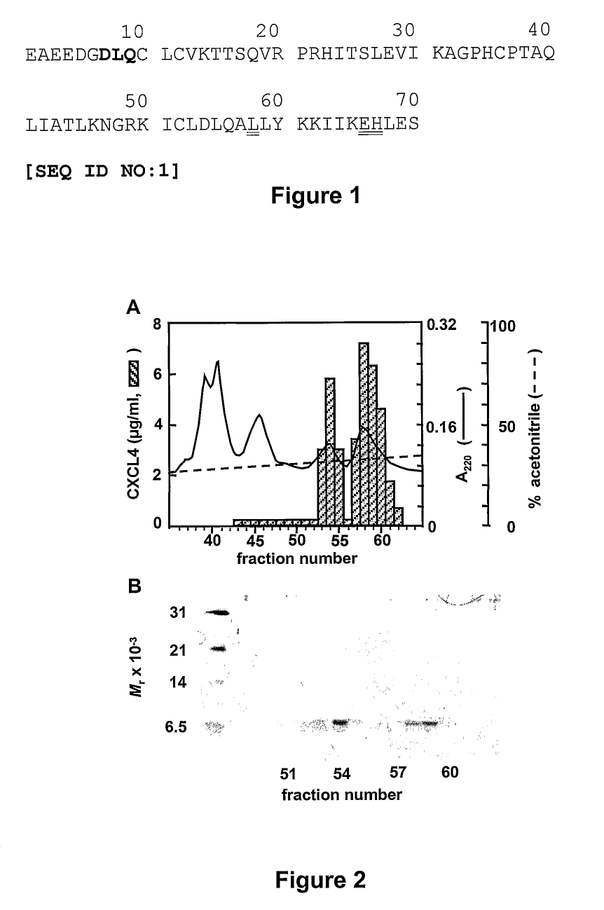

### EXAMPLES

### Example 1

### Isolation of PF4var1

### Methods

### Measurement of Human PF4 by ELISA

For the detection of human PF4, a classical sandwich ELISA was developed as described previously for other chemokines using mouse monoclonal anti-human PF4 (R&D Systems, Abingdon, UK) and rabbit polyclonal anti-human PF4 (Peprotech, Rocky Hill, N.J.) as primary and secondary antibodies, respectively [Struyf et al. (2003) Am J Pathol 163, 2065-2075]. Purified natural PF4 (vide infra) was used as a standard. This ELISA could not discriminate between different NH₂-terminal forms of PF4 and PF4var1.

Purification and Identification of PF4var1

For large scale PF4 production, outdated platelets (Red Cross blood transfusion centre, Belgium) were stimulated with 1 U/ml thrombin (Sigma, St. Louis, Mo.) for 2 h at 37° C. in PBS. The conditioned medium was subsequently purified by a purification procedure previously described [Struyf et al (2003), cited above, Van Damme et al. (1992) J Exp Med 176, 59-65; Struyf et al. (2000) in Balkwill, F. ed. Cytokine molecular biology, a practical approach. Oxford, UK, Oxford University Press, pp 73-88]. The conditioned media (1 to 5 liters) were first concentrated and partially purified by adsorption to silica (“Silica Matrex”; pore size 100 A, particle size 35-70 μm; Millipore, Billerica, Mass.; 10 g/l conditioned medium). After 2 h of magnetical stirring at 4° C., the silicic acid was sedimented by centrifugation and subsequently washed once with PBS and once with PBS containing 1 M NaCl pH 7.4. Elution of proteins from the silica matrix was done by magnetically stirring three consecutive times with 100 ml of PBS containing 1.4 M NaCl and 50% ethylene glycol pH 7.4 during 30 min at 4° C. A second elution with 0.3 M glycine/HCl pH 2.0 buffer (2×100 ml) was performed to improve recovery of chemokines. Subsequently, the pooled eluates (500 ml) were dialyzed against equilibration/loading buffer (50 mM Tris/HCl-50 mM NaCl, pH 7.4) before fractionation by heparin-Sepharose chromatography (Amersham Pharmacia Biotech, Uppsala, Sweden). Proteins were eluted from the column in a linear NaCl gradient (0.05-2 M NaCl in the loading buffer; 5 ml-fractions). For all fractions, the protein concentration was determined by a Coomassie blue G-250 binding assay using the Bio-Rad commercial kit (Bio-Rad Laboratories, Hercules, Calif.). After adjustment of the pH (from pH 7.4 to below pH 4.0 with 1% trifluoroacetic acid (TFA) in H₂O), pooled heparin-Sepharose fractions containing PF4 immunoreactivity (ELISA described above) were injected on a RP-HPLC column equilibrated with 0.01% TFA in H₂O(C-8 Aquapore RP-300 column; PerkinElmer, Norwalk, Conn. or Source 5 RPC column; Amersham Biosciences) and eluted in an acetonitrile (0-80%) gradient (0.4 or 1 ml-fractions). Sometimes an additional chromatographical step was needed to obtain homogenous chemokine preparations. In that case RP-HPLC fractions were further purified by Mono S (Amersham Pharmacia Biotech) cation-exchange fast protein liquid chromatography (FPLC). A linear NaCl (0-1 M) gradient in 50 mM formate pH 4.0 was used to elute proteins (0.2 ml-fractions). Purified fractions were analyzed by SDS-PAGE under reducing conditions to determine purity. Pure proteins were identified by NH₂-terminal amino acid sequence analysis on a Procise 491 cLC protein sequencer (Applied Biosystems, Foster City, Calif.). For mass spectrometry, RP-HPLC fractions were diluted ⅕ or 1/10 with 0.1% acetic acid in 50% acetonitrile/50% H₂O and sprayed at 5 μl/min in the source of an electrospray ESQUIRE ion trap mass spectrometer (Bruker/Daltonic, Bremen, Germany).

### Results:

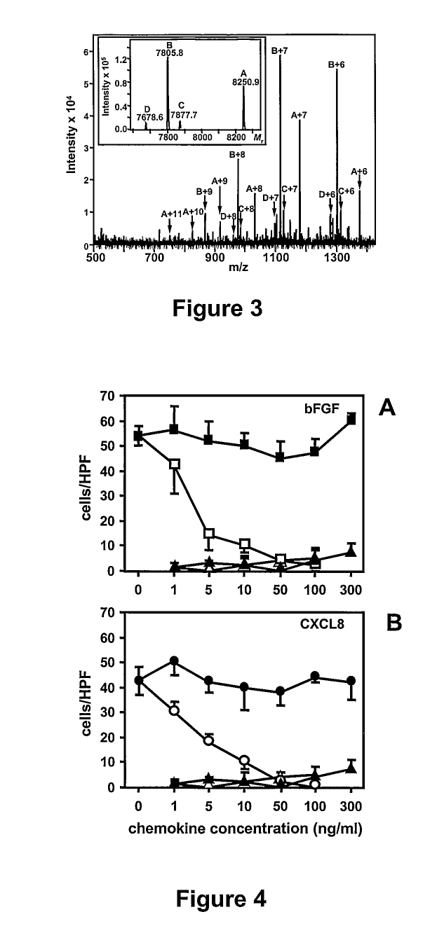

The release of chemokines from the alpha-granules of outdated platelet preparations was stimulated with thrombin. The conditioned media were concentrated and subsequently fractionated by heparin-Sepharose affinity chromatography (not shown). The major peak of PF4 immunoreactivity eluted from this column at high salt concentrations (1.6-1.8M NaCl) and contained authentic PF4 as determined by mass spectrometry. The PF4 immunoreactivity detected in fractions with intermediate heparin-affinity (eluting at 1-1.4M NaCl) was further purified by RP-HPLC and eluted in two separate peaks between 32% and 34% acetonitrile (FIG. 2A), both corresponding to pure 8 kDa protein bands on SDS-PAGE (FIG. 2B). As deduced from a combination of sequence analysis and mass spectrometry (Table 2 and FIG. 3) the RP-HPLC fractions N^{o} 57-60 contained authentic PF4. The NH₂-terminal sequence and detected average Mᵣ of the PF4 immunoreactivity (fractions N^{o} 53-55) eluting at 32% acetonitrile corresponded to PF4var1. Authentic PF4 with high affinity for heparin was purified in parallel with PF4var1 and homogenous preparations of the mature proteins were compared for angiostatic activity.

TABLE 2

Biochemical characterization of natural PF4
and PF4var1 protein shown in FIGS. 2A and B

Protein sequencing^{a}

| | | |
|---|---|---|
| | Relative | Determined |
| Chemokine | Abundancy | NH2-terminal sequence^{a} |

| | | |
|---|---|---|
| PF4var1 | 20% | FARAEAEEDGDLQXLXV |
| | | KTT |
| | 7% | [SEQ ID NO:7] |
| | | AEAEEDGDLQXLXVKTT |
| | 63% | [SEQ ID NO:8] |
| | | EAEEDGDLQXLXVKTTS |
| | 10% | QVR |
| | | [SEQ ID NO:9] |
| | | AEEDGDL |
| | | [SEQ ID NO:10] |

Mass Spectrometry^{b}

| | | | | |
|---|---|---|---|---|
| | Relative | Deduced | Experim. | |
| Chemokine | Abundancy | NH2-terminal sequence^{b} | Mᵣ | Theor. Mᵣ |

| | | | | |
|---|---|---|---|---|
| PF4var1^{c} | 30% | FARAEAEEDGDLQ . . . | 8250.9 | 8251.7 |
| | | [SEQ ID NO:11 ] | 7877.7 | 7877.2 |
| | 10% | AEAEEDGDLQ . . . | 7805.8 | 7806.1 |
| | | [SEQ ID NO:12] | 7678.6 | 7677.0 |
| | 53% | EAEEDGDLQ . . . | | |
| | | [SEQ ID NO:13] | | |
| | 7% | AEEDGDLQ . . . | | |
| | | [SEQ ID NO:14] | | |
| PF4 | 8% | SAEAEEDGDLQ . . . | 7923.8 | 7923.3 |
| | 92% | [SEQ ID NO:15] | 7764.0 | 7765.2 |
| | | EAEEDGDLQ . . . | | |
| | | [SEQ ID NO:13] | | |
| PF4 | 15% | FASAEAEEDGDLQ . . . | 8141.1 | 8141.6 |
| | 85% | [SEQ ID NO:16] | 7764.6 | 7765.2 |
| | | EAEEDGDLQ . . . | | |
| | | [SEQ ID NO:13] | | |
| PF4 | 40% | FASAEAEEDGDLQ . . . | 8141.3 | 8141.6 |
| | 60% | [SEQ ID NO:16] | 7765.6 | 7765.2 |
| | | EAEEDGDLQ. . . | | |
| | | [SEQ ID NO:13] | | |

^{a}Edman degradation (determined NH₂-terminal sequence) and
^{b}mass spectrometry analysis results (deduced NH₂-terminal sequence (in italic) and experimental Mᵣ) of C8 RP-HPLC purified natural PF4var1 and PF4 (also shown in FIG. 2).
^{c}PF4var1 was present in different molecular forms: a protein containing one (A) and four (FARA) extra NH₂-terminal residues from the signal peptide, the predicted mature protein (EAEEDG . . .) [SEQ ID NO:18] and a shorter protein missing the NH₂-terminal glutamine (AEEDG . . .) [SEQ ID NO:19].
Biological characterization was performed with fractions containing predominantly the mature PF4var1 protein (EAEEDG . . .) [SEQ ID NO:18] in comparison with mature PF4 (EAEEDG . . .) [SEQ ID NO:18] obtained after the same purification procedure starting from the major PF4 immunoreactivity peak with high affinity for heparin.

### Example 2

### Endothelial Cell Chemotaxis

### In Vitro Assay:

Human lung microvascular endothelial cells (HMVEC-L) were obtained from Clonetics (Walkersville, Md.), and were cultured following the manufacturer's instructions in EBM-2 medium supplemented with the EGM-2-MV “bulletkit” containing FCS, ascorbic acid, hydrocortisone, fibroblast growth factor (FGF), vascular endothelial growth factor (VEGF), insulin-like growth factor I (IGF-I) and epidermal growth factor (EGF). Endothelial cell chemotaxis assays were performed as previously described [Addison et al. (2002) J Immunol 165, 5269-5277]. Cells were harvested by trypsinization, resuspended in EBM-2 medium without growth factors with 2% FCS added. An aliquot of 160 μl containing 5×10⁵ cells/ml was added to each of the lower wells of a 12-well chemotaxis chamber (Neuro Probe Inc., Cabin John Md.). The chambers were assembled (by placing 0.1% gelatin coated 5 μm pore size filters over the lower wells, followed by a gasket and the upper chamber) and incubated at 37° C. in a CO₂ incubator for 2 h in an inverted position. The chambers were then turned upright, and 100 μl aliquots of sample dilutions were added to the upper wells of the chamber. As endothelial cell chemoattractants CXCL8 and bFGF (both from Peprotech) were used. The chambers were placed in a 37° C. CO₂ incubator for 2 h at which time the filters were removed, and subjected to Diff-Quik staining. The total number of migrated cells was counted in 15 separate fields of view under 400× power. Results were expressed as the number of endothelial cells that migrated per high power field (HPF) after subtracting the background (unstimulated control) to demonstrate specific migration.

### Rat Corneal Micropocket Assay for Angiogenesis

In vivo angiogenesis was assessed using the rat cornea micropocket assay as previously described [Addison et al. cited above]. Briefly, reagents were diluted in PBS plus 0.25% serum albumin to their final concentration per pellet of Hydron casting solution (Hydro Med Sciences, New Brunswick N.J.). 5 μl aliquots were pipetted onto the flat surface of a sterile polypropylene specimen container and were allowed to polymerize overnight under UV light in a laminar flow hood. Prior to implantation, the pellets were rehydrated with normal saline. Animals were given 150 mg/kg ketamine and 250 μg/kg atropine, and the corneas were anesthetized with 0.5% proparacaine hydrochloride ophthalmic solution followed by implantation of the Hydron pellet into an intracorneal pocket (1-2 mm from the limbus). Six days after implantation, animals received heparin (1000 U) and ketamine (150 mg/kg) i.p. 30 min prior to sacrifice, followed by perfusion with 10 ml of colloidal carbon via the left ventricle. Corneas were then harvested and photographed. No inflammatory response was observed in any of the corneas treated with the cytokines tested. Sustained directional ingrowth of capillary sprouts and hairpin loops toward the implant were considered positive neovascularization responses. Negative responses were characterized by either no vessel growth or by the presence of only an occasional hairpin loop or sprout that displayed no evidence of sustained growth.

### Results:

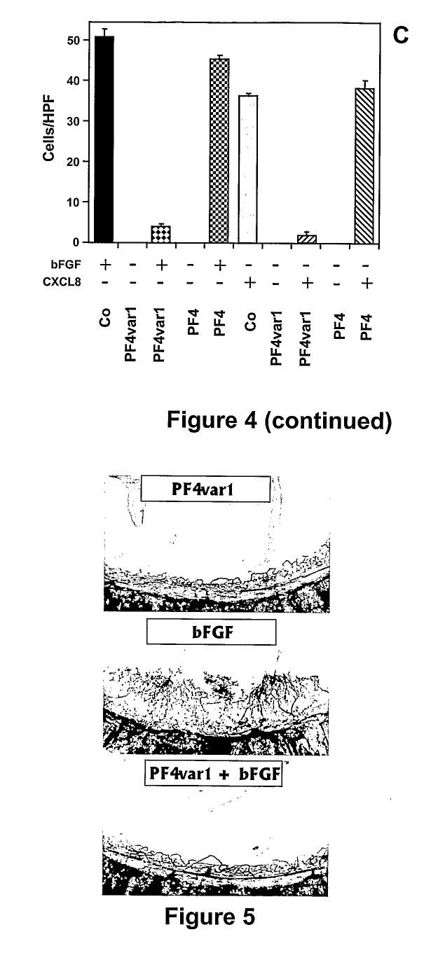

bFGF (50 ng/ml) and CXCL8 (80 ng/ml) induced HMVEC chemotaxis (FIGS. 4 A and B) which was completely blocked by PF4var1 (10-100 ng/ml). In contrast, even at 300 ng/ml authentic PF4 failed to inhibit bFGF- or CXCL8-induced chemotaxis. Subsequently, the cornea micropocket assay was used to determine whether PF4var1 inhibits angiogenesis in vivo. As shown in FIG. 5 and Table 3, PF4var1 (50 ng) inhibited bFGF (50 ng) in 16 of 22 corneas, representing a 73% inhibition of angiogenesis. In contrast, at the same dose authentic PF4 inhibited bFGF in only 5 of 22 corneas.

TABLE 3
In vivo angiostatic activity of PF4var1

| | N^{o} of pos | N^{o} of | |
|---|---|---|---|
| Treatment | pellets^{a} | neg pellets^{a} | % inhibition^{b} |
| buffer (vehicle control) | 0/5 | 5/5 | 0% |
| bFGF (50 ng) | 25/25 | 0/25 | NA^{c} |
| PF4 (50 ng) | 5/22 | 17/22 | NA |
| PF4 (50 ng) + bFGF (50 ng) | 17/22 | 5/22 | 23% |
| PF4var1 (50 ng) | 1/22 | 21/22 | NA |
| PF4var1 (50 ng) + bFGF (50 ng) | 6/22 | 16/22 | 73% |

^{a}Rat cornea neovascularization in response to 50 ng of PF4, PF4var1, bFGF, or combinations of these cytokines. Numbers indicate the number of pellets (“positive pellets”) in which an angiogenic response was observed (for criteria see materials and methods section) or “negative pellets” in which no neovascularization occurred, relative to the total number of pellets implanted.
^{b}The percentage inhibition of bFGF-induced angiogenesis in the pellets in which PF4 or PF4var1 was combined with bFGF.
^{c}NA, not applicable.

These data demonstrate that PF4var1 is an angiostatic factor stored in platelets, which undergo degranulation at sites of vascular injury and/or thrombosis and modulate net angiogenesis within the local microenvironment. The examples of the present invention further show that PF4var1, as compared to PF4, is a more potent inhibitor of endothelial cell chemotaxis in vitro and has more profound effects in blocking angiogenesis in vivo. These results show that PF4var1 can be used as a therapeutic tool to inhibit aberrant angiogenesis in a variety of diseases.

### Example 3

### In Vivo Effect of PF4var1 on Pathological Angiogenesis: Intratumoral Injection of B16 Melanoma Tumors with Recombinant Human PF4var1

Animal experiments were approved by the local animal ethics committee (University of Leuven), and conducted in conformity with the Belgian and European guidelines for the protection of animals used for scientific purposes. Experiments were performed with 6 to 8 weeks old female athymic nu/nu mice (NMRI background) kept in a specific pathogen-free environment (Elevage Janvier, Le Genest Saint Isle, France). Two consecutive experiments were performed, the first comprising 20 mice, the second 18 mice. B16 melanoma cells in log phase (2×10⁶ cells resuspended in 200 μl of PBS) were injected subcutaneously on day 1 in the right dorsal flank. Animals were injected at the tumor site with 50 μl of test sample, i.e. endotoxin-free saline (0.9% NaCl, Baxter, Lessines, Belgium), 5 pg of natural human PF4 or 1 μg of recombinant human PF4var1 in saline. Treatment started on day 3, in total 6 intratumoral injections were administered three times a week over a period of 2 weeks. All animals were observed three times a week, the tumor dimensions were measured with calipers and tumor size was calculated (tumor volume=4/3Πab²; a is the smallest diameter/2 and b is the largest diameter/2 in millimeters). At the end of the experiments tumor size was calculated more precisely after dissection of the tumor using the formula 4/3Πabc (a, b and c width/2, length/2 and depth/2, respectively). In the second experiment dissected tumors were weighed as well.

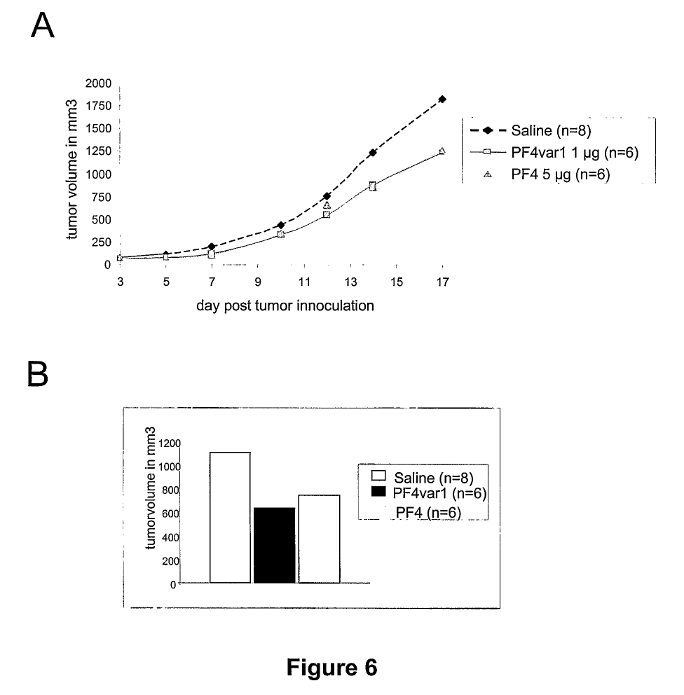

The results (FIG. 6, A, B) demonstrate that PF4var1 is capable of reducing tumor growth.

1-27. (canceled)

28. A method of treating a mammal suffering from or at risk of developing a disease involving an angiogenic disorder, comprising the step of administering to a mammal an effective amount of a protein or peptide comprising an amino acid sequence corresponding to the sequence Leu-Xaa-Xaa-Xaa-Xaa-Xaa-Xaa-Xaa-Glu-His [SEQ ID NO: 4] and having at least 60% sequence similarity to Leu-Leu-Tyr-Lys-Lys-Ile-Ile-Lys-Glu-His [SEQ ID NO: 5] thereby inhibiting angiogenesis in said mammal.

29. The method according to claim 28, wherein said disease is cancer.

30. The method according to claim 29, wherein said cancer is cancer of the prostate, lung, breast, bladder, kidney, pancreas, liver, ovaries, uterus, stomach or is a metastatic skin or rectal tumor.

31. The method according to claim 28, wherein said disease is an eye-disease.

32. The method according to claim 28, wherein said protein or peptide comprises the sequence Leu-Leu-Tyr-Lys-Lys-Ile-Ile-Lys-Glu-His depicted in SEQ ID NO: 5.

33. The method according to claim 28, wherein said protein or peptide comprises the sequence Leu-Leu-Tyr-Lys-Lys-Ile-Ile-Lys-Glu-His-Leu-Glu-Ser depicted in SEQ ID NO: 3.

34. The method according to claim 28, wherein said fragment is a c-terminal fragment of PF4var1, or a fragment having at least 70% sequence identity therewith.

35. The method according to claim 28, wherein said protein or peptide comprises the sequence of the PF4var1⁴⁷⁻⁷⁰ fragment.

36. The method according to claim 28, wherein the said protein comprises the sequence of the PF4var1⁶⁸⁻⁷⁰ fragment.

37. The method according to claim 28, wherein said protein comprises the sequence of PF4var1 depicted in SEQ ID NO:1.

38. The method according to claim 28, wherein said protein or peptide comprises the sequence Leu-Leu-Tyr-Gln-Glu-Ile-Ile-Gln-Glu-His [SEQ ID NO: 6].

39. The method according to claim 28, wherein said peptide comprises the sequence Leu-Leu-Tyr-Gln-Glu-Ile-Ile-Gln-Glu-His-Leu-Glu-Ser [SEQ ID NO: 20].

40. The method according to claim 28, wherein said peptide is selected from the group consisting of:
a peptide consisting of the sequence Leu-Leu-Tyr-Lys-Lys-Ile-Ile-Lys-Glu-His depicted in SEQ ID NO: 5;
a peptide consisting of the sequence Leu-Leu-Tyr-Lys-Lys-Ile-Ile-Lys-Glu-His-Leu-Glu-Ser depicted in SEQ ID NO: 3;
a peptide consisting of the sequence of the PF4var1⁴⁷⁻⁷⁰ fragment;
a peptide consisting of the sequence of the PF4var1⁶⁸⁻⁷⁰ fragment;
a peptide consisting of the sequence Leu-Leu-Tyr-Gln-Glu-Ile-Ile-Gln-Glu-His [SEQ ID NO: 6];
a peptide consisting of the sequence Leu-Leu-Tyr-Gln-Glu-Ile-Ile-Gln-Glu-His-Leu-Glu-Ser depicted in SEQ ID NO: 20; and
a protein consisting of the sequence of PF4var1 depicted in SEQ ID NO:1.

41. A process for obtaining PF4var1 or fragments thereof comprising the steps of a) lysing platelets or stimulating platelets in a medium under conditions whereby the alpha-granules of the platelets are released in said medium and b) purifying PF4var1 from said medium based on intermediate heparin-affinity, and optionally c) obtaining fragments from said PF4var1.

42. The process of claim 41, comprising stimulating said platelets with thrombin.

43. A protein or peptide consisting of between 10 and 70 amino acids and comprising the sequence Leu-Xaa-Xaa-Xaa-Xaa-Xaa-Xaa-Xaa-Glu-His [SEQ ID NO: 4] having at least 60% sequence similarity to the sequence Leu-Leu-Tyr-Lys-Lys-Ile-Ile-Lys-Glu-His [SEQ ID NO: 5].

44. The protein or peptide according to claim 43, which comprises the sequence Leu-Leu-Tyr-Lys-Lys-Ile-Ile-Lys-Glu-His depicted in SEQ ID NO: 5.

45. The protein or peptide according to claim 43, which comprises the sequence Leu-Leu-Tyr-Lys-Lys-Ile-Ile-Lys-Glu-His-Leu-Glu-Ser depicted in SEQ ID NO: 3.

46. The protein or peptide according to claim 43, which is a c-terminal fragment of PF4var1, or a fragment having at least 70% sequence identity therewith.

47. The protein or peptide according to claim 43, which comprises the sequence of the PF4var1⁴⁷⁻⁷⁰ fragment.

48. The protein or peptide according to claim 43, which comprises the sequence of the PF4var1⁶⁸⁻⁷⁰ fragment.

49. The protein or peptide according to claim 43, which comprises the sequence Leu-Leu-Tyr-Gln-Glu-Ile-Ile-Gln-Glu-His [SEQ ID NO: 6].

50. The protein or peptide according to claim 43, which comprises the sequence Leu-Leu-Tyr-Gln-Glu-Ile-Ile-Gln-Glu-His-Leu-Glu-Ser [SEQ ID NO: 20].

51. The protein or peptide according to claim 43, which is selected from the group consisting of:
a peptide consisting of the sequence Leu-Leu-Tyr-Lys-Lys-Ile-Ile-Lys-Glu-His depicted in SEQ ID NO: 5;
a peptide consisting of the sequence Leu-Leu-Tyr-Lys-Lys-Ile-Ile-Lys-Glu-His-Leu-Glu-Ser depicted in SEQ ID NO: 3;
a peptide consisting of the sequence of the PF4var1⁴⁷⁻⁷⁰ fragment;
a peptide consisting of the sequence of the PF4var1⁶⁸⁻⁷⁰ fragment;
a peptide consisting of the sequence Leu-Leu-Tyr-Gln-Glu-Ile-Ile-Gln-Glu-His [SEQ ID NO: 6]; and
a peptide consisting of the sequence Leu-Leu-Tyr-Gln-Glu-Ile-Ile-Gln-Glu-His-Leu-Glu-Ser depicted in SEQ ID NO: 20.


SEQUENCE LISTING (28 sequences)

SEQ ID NO: 1 - protein, 70 aa, Homo sapiens
  mat_peptide (1)..(70): PFvar1
EAEEDGDLQCLCVKTTSQVRPRHITSLEVIKAGPHCPTAQLIATLKNGRKICLDLQALLYKKIIKEHLES

SEQ ID NO: 2 - protein, 13 aa, artificial sequence
  PF4var1 carboxyterminal fragment derived peptide
LXXXXXXXEHLES

SEQ ID NO: 3 - protein, 13 aa, artificial sequence
  PF4var1 carboxyterminal fragment (AA 58-70)
LLYKKIIKEHLES

SEQ ID NO: 4 - protein, 10 aa, artificial sequence
  PF4var1 LX7EH sequence motif
LXXXXXXXEH

SEQ ID NO: 5 - protein, 10 aa, artificial sequence
  PF4var1 carboxyterminal fragment (AA 58-67)
LLYKKIIKEH

SEQ ID NO: 6 - protein, 10 aa, artificial
  modified PF4var1 carboxyterminal fragment (AA 58-67)
LLYQEIIQEH

SEQ ID NO: 7 - protein, 20 aa, artificial sequence
  PF4var1 aminoterminal fragment  (AA -4 - 16)
FARAEAEEDGDLQXLXVKTT

SEQ ID NO: 8 - protein, 17 aa, artificial sequence
  PF4var1 aminoterminal fragment (AA -1 - 16)
AEAEEDGDLQXLXVKTT

SEQ ID NO: 9 - protein, 20 aa, artificial sequence
  PF4var1 aminoterminal fragment (AA 1 - 20)
EAEEDGDLQXLXVKTTSQVR

SEQ ID NO: 10 - protein, 7 aa, artificial sequence
  PF4var1 aminoterminal fragment (AA 2 - 8)
AEEDGDL

SEQ ID NO: 11 - protein, 13 aa, artificial sequence
  PF4var1 aminoterminal fragment (AA -4 - 9)
FARAEAEEDGDLQ

SEQ ID NO: 12 - protein, 10 aa, artificial sequence
  PF4var1 aminoterminal fragment (AA -1 - 9)
AEAEEDGDLQ

SEQ ID NO: 13 - protein, 9 aa, artificial sequence
  PF4var1 aminoterminal fragment (AA 1 - 9)
EAEEDGDLQ

SEQ ID NO: 14 - protein, 8 aa, artificial sequence
  PF4var1 aminoterminal fragment (AA 3 - 9)
AEEDGDLQ

SEQ ID NO: 15 - protein, 11 aa, artificial sequence
  PF4 aminoterminal fragment (AA -2 - 9)
SAEAEEDGDLQ

SEQ ID NO: 16 - protein, 13 aa, artificial sequence
  PF4 aminoterminal fragment (AA -4 - 9)
FASAEAEEDGDLQ

SEQ ID NO: 17 - protein, 4 aa, artificial sequence
  PF4var1 aminoterminal fragment (AA -4 -1)
FARA

SEQ ID NO: 18 - protein, 6 aa, artificial sequence
  PF4var1 aminoterminal fragment (AA 1 -  6)
EAEEDG

SEQ ID NO: 19 - protein, 5 aa, artificial sequence
  PF4var1 aminoterminal fragment (AA  2 - 6)
AEEDG

SEQ ID NO: 20 - protein, 13 aa, artificial peptide
  PF4var1 carboxyterminal fragment (AA 58-70) modified version
LLYQEIIQEHLES

SEQ ID NO: 21 - protein, 8 aa, artificial sequence
  PFvar1 X6EH sequence motif
XXXXXXEH

SEQ ID NO: 22 - protein, 10 aa, artificial sequence
  PFvar1 L-[VILM]-[YFW]-[KHR]-[KHR]-[VILM]- [VILM]-[KHR]-E-H motif
LXXXXXXXEH

SEQ ID NO: 23 - protein, 9 aa, artificial sequence
  PFvar1 XKKXXKEH motif
XKKXXKKEH

SEQ ID NO: 24 - protein, 10 aa, artificial sequence
  PFvar1 LXXKKXXKEH motif
LXXKKXXKEH

SEQ ID NO: 25 - protein, 9 aa, artificial sequence
  PFvar1 LX6EH motif
LXXXXXXEH

SEQ ID NO: 26 - protein, 9 aa, artificial sequence
  PFvar1 X7EH motif
XXXXXXXEH

SEQ ID NO: 27 - protein, 7 aa, artificial sequence
  PFvar1 KKXXKEH motif
KKXXKEH

SEQ ID NO: 28 - protein, 7 aa, artificial sequence
  PFvar1 XXIIXEH motif
XXIIXEH